(19) United States
(12) Patent Application Publication
(10) Pub. No.: US 2024/0033128 A1
(43) Pub. Date: Feb. 1, 2024

(54) HOLLOWED-OUT EARPICK HEAD AND EARPICK

(71) Applicant: SHENZHEN NAN BO ZHI JU TECHNOLOGY CO., LTD, Shenzhen (CN)

(72) Inventors: Qiang QIN, Shenzhen City (CN); Shan GAO, Shenzhen City (CN)

(21) Appl. No.: 18/376,109

(22) Filed: Oct. 3, 2023

Related U.S. Application Data
(63) Continuation of application No. PCT/CN2021/101187, filed on Jun. 21, 2021.

(30) Foreign Application Priority Data
Apr. 12, 2021 (CN) 202110390137.2

(51) Int. Cl. A61F 11/00

(52) U.S. Cl. CPC A61F 11/006

(57) ABSTRACT

A hollowed-out earpick head and an earpick, belonging to the field of ear canal cleaning, includes a connecting rod and a cleaning end on an end surface at one end of the connecting rod, an outer wall of the cleaning end is an outwardly convex arc-shaped wall, the cleaning end is hollowed out with a pre-storage cavity, a transportation cavity in communication with the pre-storage cavity extends through the connecting rod, an end of the cleaning end away from the connecting rod is configured with a collecting tank in communication with the pre-storage cavity, the cleaning end is hollowed out with a lateral groove. The cleaning end is hollowed out and in communication with the pre-storage cavity.

### CROSS-REFERENCE TO RELATED APPLICATIONS

This application is a continuation of PCT application serial no. PCT/CN2021/101187, filed on Jun. 21, 2021, which claims the priority and benefit of Chinese patent application serial no. 202110390137.2, filed on Apr. 12, 2021. The entirety of PCT application serial no. PCT/CN2021/101187 and Chinese patent application serial no. 202110390137.2 are hereby incorporated by reference herein and made a part of this specification.

### TECHNICAL FIELD

The present application relates to the technical field of ear canal cleaning, and in particular, to a hollowed-out earpick head and an earpick.

### BACKGROUND ART

The skin of the external meatal cartilage of a human body has ceruminous glands, whose secretions are mixed with the sebum discharged from the sebaceous glands together to form a very thin layer attached to the surface of the skin, i.e., earwax. Most of the earwax is often self-discharged with the movement of the mandibular joint with the aid of chewing, mouth opening and other movements. The earwax would be mixed with dust and dandruff to form cerumen over time, bacteria are easy to breed, and even irritate the skin of the external auditory meatus, which leads to inflammation, so it is necessary to regularly clean the ears.

People often use ear pickers (earpicks) to clean the external auditory meatus. Existing ear pickers have a relatively large head, by which it is necessary to deliver the head of the ear picker into the external auditory meatus from outside to inside. However, part of the earwax is in the middle of the ear canal in a granular state. When cleaning the ear canal with an earpick, the granular earwax would be pushed deep into the ear easily, which may cause accumulation of the earwax in the external ear, such that the user may feel painful and suffer from infection in the long run, which is required to be further improved.

### SUMMARY

In order to reduce the possibility that the granular earwax in the ear canal is pushed towards the outer ear during cleaning of the ear canal, a hollowed-out earpick head and an earpick are disclosed.

In a first aspect, a hollowed-out earpick head is disclosed, which includes a connecting rod and a cleaning end on an end surface of one end of the connecting rod, the cleaning end is hollowed out with a pre-storage cavity, the connecting rod is configured with a transportation cavity in communication with the pre-storage cavity, a first end of the cleaning end away from the connecting rod is configured with a collecting tank in communication with the pre-storage cavity, and the cleaning end is hollowed out and in communication with the transportation cavity.

By using the above-mentioned technical solution, when the earpick head is used to remove the earwax in the ear canal, the cleaning end is slowly pushed into the interior of the ear canal by means of the connecting rod and hereby the cleaning end is driven to rotate by the connecting rod. During pushing the cleaning end, the granular earwax in the ear canal enters the pre-storage cavity through the opening of the collecting tank. In addition, the earpick can collect the earwax entering the pre-storage cavity in the earpick through the transportation cavity by means of negative pressure adsorption, playing a role of sending the earwax out from the pre-storage cavity in time. Through the above-mentioned process, when removing the earwax, the whole cleaning end of the earpick head is hollowed out, through which an inner wall of the ear canal is not damaged in combination with an arc-shaped wall of the cleaning end, so that the possibility that the granular earwax in the ear canal is pushed to an outer ear during the ear canal cleaning process can be reduced.

Alternatively, the cleaning end is hollowed out with a lateral groove in communication with an outer wall of the pre-storage cavity, and a central axis of the collecting tank is not collinear with a central axis of the connecting rod.

By using the above-mentioned technical solution, an inner wall of the lateral groove along a length direction of the connecting rod can scrape off the earwax adhered to the inner wall of the ear canal, such that the earwax on the inner wall of the ear canal can be removed. When the earpick head is slowly pushed deep into the ear canal, the cleaning end can be rotated at the same time to drive the opening of the collecting tank to rotate. During the rotation of the opening direction of the collecting tank, the granular earwax from multiple directions in the ear canal can be sent to the pre-storage cavity to perform the function of multi-directional cleaning.

Alternatively, a protrusion is formed on an inner wall at a side of the lateral groove facing the connecting rod, and an end of the protrusion facing the connecting rod is an arc surface.

By using the above-mentioned technical solution, it is unnecessary to rotate the cleaning end by rotating the connecting rod during using the earpick head, and the function of a normal earpick can be achieved with the protrusion.

Alternatively, the first end of the cleaning end away from the connecting rod is a round head, and the round head has a cross section tapering in a direction away from the connecting rod and perpendicular to a length direction of the connecting rod.

By the above-mentioned technical solution, the end of the cleaning end away from the connecting rod is a round head with a cross section gradually tapering in a direction away from the connecting rod and perpendicular to a length of the connecting rod, which plays a guiding role when the cleaning head is slowly pushed into the ear canal. In addition, the contact area between the section of the cleaning end without the opening of the collecting tank and the earwax in the ear canal is reduced, so that the possibility that the granular earwax in the ear canal is pushed to the outer ear during the cleaning process of the ear canal is further reduced.

Alternatively, a distance between inner walls on opposite sides of the lateral groove gradually increases towards a central axis of the pre-storage cavity.

When the inner wall of the lateral groove scrapes off the earwax adhering to the inner wall of the ear canal, the cleaning end continuously rotates, the inner wall of the lateral groove will block some of the scraped earwax, which hinders the scraped earwax to enter the pre-storage cavity, such that the earwax cannot be sent out in time. By the above-mentioned technical solution, the distance between the inner walls at the opposite sides of the lateral groove gradually increase in the direction close to the central axis of the pre-storage cavity, in this case, the inner wall of the lateral groove is obliquely relative to the opening of the lateral groove, by which the scraped earwax can be slowly guided into the pre-storage cavity when the cleaning end continuously rotates.

Alternatively, a mounting sleeve is attached to a second end of the connecting rod away from the cleaning end and has a diameter greater than a diameter of the connecting rod, the mounting sleeve is configured with a connecting slot in communication with the transportation cavity, and the connecting slot extends through the transportation cavity.

During the use of the earpick head, the earpick head must be assembled with other fittings of the earpick. The assembly of the whole earpick head can be facilitated with the mounting sleeve in the above-mentioned technical solution, and the connecting slot allows the transportation cavity to be in communication with the adsorption structure on the earpick.

In a second aspect, an earpick is further disclosed, including a housing and the hollowed-out earpick head, wherein an inner cavity is defined in the housing, an end of the housing close to the earpick head is configured with a mounting port, a rotary sleeve configured for inserting in the connecting slot is provided in the mounting port, an outer wall of the rotary sleeve is circumferentially fixed relative to an inner wall of the connecting slot, an adsorption mechanism configured for adsorbing air in a transportation cavity is provided in the inner cavity, and a driving mechanism configured for driving the rotary sleeve to rotate is provided in the inner cavity.

By using the above-mentioned technical solution, during the use of the earpick, the earpick head can be sleeved on the rotary sleeve with the mounting port and the rotary sleeve, hereby the rotary sleeve is embedded in the connecting slot, and the outer wall of the rotary sleeve is in an interference fit with the inner wall of the connecting slot. In the process of pushing the earpick head into the ear canal, the rotary sleeve can be driven to rotate in the ear canal by the driving mechanism. In the process of pushing the cleaning end, the granular earwax in the ear canal enters the pre-storage cavity through the opening of the collecting tank, and rotation of the cleaning end in the ear canal drives the opening direction of the collecting tank to change in the ear canal. During rotation of the opening direction of the collecting tank, the granular earwax from multiple directions in the ear canal can be sent to the pre-storage cavity. When the earwax in the ear canal at the collection port is collected in the pre-storage cavity, a negative pressure can be formed between the rotary sleeve, the connecting slot and the transportation cavity by means of the adsorption mechanism, so that the earwax can be delivered. During removing the earwax, the possibility that the granular earwax in the ear canal is pushed to the outer ear in the process of cleaning the ear canal can be reduced by the above process.

Alternatively, the adsorption mechanism includes an electric motor in the inner cavity, a rotary shaft at an output shaft of the electric motor, a rotary disk at an end of the rotary shaft away from the electric motor, and a fan blade on an end of the rotary disk away from the rotary shaft, a limit portion is provided in the inner cavity and is configured with a negative pressure cavity configured for rotation of the rotary disk and the fan blade to form a negative pressure, air circulates between an end of the negative pressure cavity close to the earpick head and the rotary sleeve, an inner wall at a side of the negative pressure cavity away from the rotary sleeve is configured with a vent hole, and an outer wall of the housing is configured with a vent in communication with the inner cavity.

By using the above-mentioned technical solution, when the earpick is used, the electric motor drives the rotary disk and the fan blade to rotate, hereby the air in the negative pressure cavity is discharged out of the housing through the vent hole and the vent, meanwhile, the air in the rotary sleeve and the transportation cavity is also discharged, and the inside of the negative pressure cavity, the rotary sleeve and the transportation cavity are continuously in a negative pressure state. After the collection port collects the earwax in the ear canal in the pre-storage cavity, the earwax is delivered out of the ear canal to remove the earwax.

Alternatively, a side of the limit portion close to the earpick head is configured with a mounting groove, the rotary sleeve is inserted into the negative pressure cavity through the mounting groove, a limit groove is defined on a side wall of the mounting groove, a first bearing is provided in the limit groove, the rotary sleeve extends through the first bearing, a limit ring is provided on an outer wall of the rotary sleeve at the mounting groove, and the limit ring abuts against a side of the first bearing away from the electric motor.

By using the above-mentioned technical solution, when the rotary sleeve is assembled, one end of the rotary sleeve extends through the mounting port and is embedded in the negative pressure cavity through the mounting groove, meanwhile, the rotary sleeve extends through the first bearing, and the limit ring on the rotary sleeve abuts against the first bearing to play the role of limiting the rotary sleeve, and play the role of assembling the rotary sleeve in the limit groove.

Alternatively, a collecting sleeve is provided in an end of the rotary sleeve away from the negative pressure cavity, a grid in form of a filter mesh is provided at an end port of a first end of the collecting sleeve close to the negative pressure cavity, an outer wall at a second end of the collecting sleeve away from the grid is provided with a convex edge, and the convex edge is configured to abut against an end surface at the end of the rotary sleeve away from the negative pressure cavity.

By using the above-mentioned technical solution, during the use of an earpick, the earwax removed by the earpick head can be collected through the collecting sleeve, the earwax in the pre-storage cavity is delivered to the collecting sleeve through the transportation cavity by the adsorption mechanism. The air circulation between the earpick head and the housing is not affected through the grid, and the removed earwax can be stored in the collecting sleeve, which can be subsequently cleaned.

In summary, at least one of the following beneficial technical effects can be achieved:

When the earpick head is used to remove the earwax in the ear canal, the cleaning end is slowly pushed into the interior of the ear canal by means of the connecting rod and hereby the cleaning end is driven to rotate by the connecting rod. During pushing the cleaning end, the granular earwax in the ear canal enters the pre-storage cavity through the opening of the collecting tank. An inner wall of the lateral groove along a length direction of the connecting rod can scrape off the earwax adhered to the inner wall of the ear canal, such that the earwax on the inner wall of the ear canal can be removed, which can cooperate with the whole earpick in use. In addition, the earpick can collect the earwax entering the pre-storage cavity in the earpick through the transportation cavity by means of negative pressure adsorption, playing a role of sending the earwax out from the pre-storage cavity in time. Through the above-mentioned process, when removing the earwax, the whole cleaning end of the earpick head is hollowed out, through which an inner wall of the ear canal is not damaged in combination with an arc-shaped wall of the cleaning end, so that the possibility that the granular earwax in the ear canal is pushed to an outer ear during the ear canal cleaning process can be reduce.

During the use of the earpick, the earpick head can be sleeved on the rotary sleeve with the mounting port and the rotary sleeve, hereby the rotary sleeve is embedded in the connecting slot, and the outer wall of the rotary sleeve is in an interference fit with the inner wall of the connecting slot. In the process of pushing the earpick head into the ear canal, the rotary sleeve can be driven to rotate in the ear canal by the driving mechanism. In the process of pushing the cleaning end, the granular earwax in the ear canal enters the pre-storage cavity through the opening of the collecting tank, and rotation of the cleaning end in the ear canal drives the opening direction of the collecting tank to change in the ear canal. During rotation of the opening direction of the collecting tank, the granular earwax from multiple directions in the ear canal can be sent to the pre-storage cavity. When the earwax in the ear canal at the collection port is collected in the pre-storage cavity, a negative pressure can be formed between the rotary sleeve, the connecting slot and the transportation cavity by means of the adsorption mechanism, so that the earwax can be delivered. During removing the earwax, the possibility that the granular earwax in the ear canal is pushed to the outer ear in the process of cleaning the ear canal can be reduced by the above process.

### BRIEF DESCRIPTION OF THE DRAWINGS

FIG. 1 is an overall structural schematic diagram showing a cleaning end and a connecting rod in the hollowed-out earpick head according to the present application;

FIG. 2 is a diagram showing the internal structure of the hollowed-out earpick head according to the present application for showing a transportation cavity;

FIG. 3 is a schematic diagram showing the partial structure of a cleaning end and a connecting rod in the hollowed-out earpick head according to the present application for showing the protrusion;

FIG. 4 is a schematic diagram showing the overall structure of an earpick according to Embodiment I of earpick of the present application;

FIG. 5 is a schematic diagram showing the structure of an earpick with a part of a housing being hidden according to Embodiment I of earpick of the present application;

FIG. 6 is a schematic diagram showing the structure of the earpick head, the rotary sleeve and the adsorption mechanism according to Embodiment I of earpick of the present application;

FIG. 7 is an exploded view showing the earpick head, the collecting sleeve and the rotary sleeve according to Embodiment I of earpick of the present application; and

FIG. 8 is a schematic diagram showing the structure of the first gear, the second gear and the third gear according to Embodiment II of earpick of the present application.

### DETAILED DESCRIPTION

This application is described in further detail below.

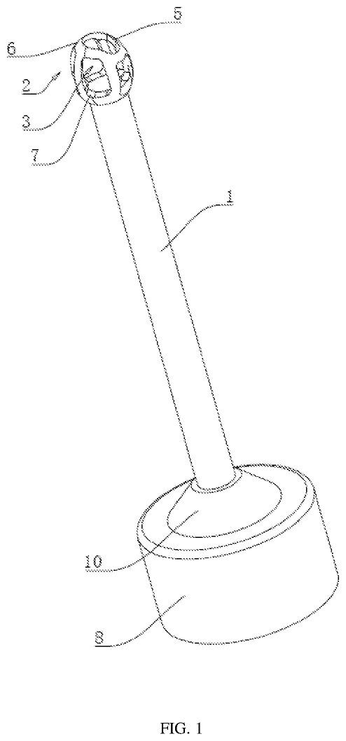

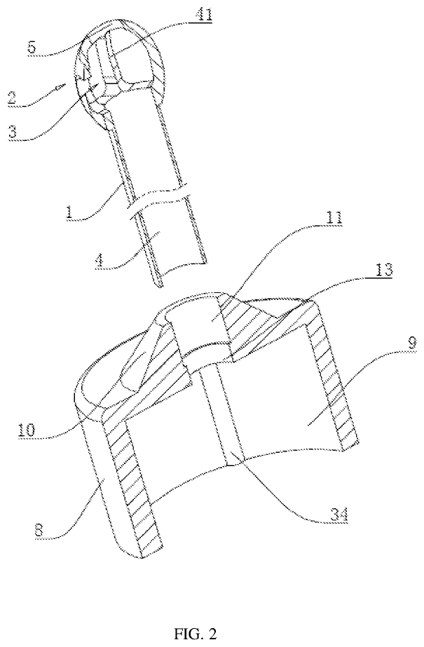

The embodiments of the present application disclose a hollowed-out earpick head with reference to FIGS. 1 and 2, the earpick head includes a connecting rod 1 and a cleaning end 2 integrally formed on an end surface at one end of the connecting rod 1, wherein an outer wall of the cleaning end 2 is an outwardly convex arc-shaped wall, and the cleaning end 2 is hollowed out with a pre-storage cavity 3, the pre-storage cavity 3 is configured for storing earwax. A transportation cavity 4 in communication with the pre-storage cavity 3 extends through the connecting rod 1, and the transportation cavity 4 is configured for delivering the earwax in the pre-storage cavity 3 in time. In order to collect and remove the granular earwax in the ear canal, a collecting tank 5 in communication with the pre-storage cavity 3 is defined at the end of the cleaning end 2 away from the connecting rod 1, wherein a central axis of the collecting tank 5 is not collinear with the central axis of the connecting rod 1, and the collecting tank 5 is defined at the end surface of the end of the cleaning end 2 away from the connecting rod 1.

The earwax in the ear canal is transported into the pre-storage cavity 3 via the opening of the collecting tank 5, while the cleaning end 2 is driven to rotate via the connecting rod 1, such that the granular earwax in the ear canal enters into the pre-storage cavity 3 through the opening of the collecting tank 5 during the advancement of the cleaning end, and the opening direction of the collecting tank 5 changes in the ear canal during the rotation of the cleaning end 2 in the ear canal, and the granular earwax from multiple directions in the ear canal can be sent to the pre-storage cavity 3 during the rotation of the opening direction of the collecting tank 5. At the same time, the earwax is sent out of the ear canal by means of the low-pressure adsorption function of the earpick in time, which reduces the possibility that the granular earwax in the ear canal will be pushed towards the outer ear during the ear canal is cleaned.

An earpick head is assembled with an earpick in use, and it is necessary to rotate the cleaning end 2 and slowly insert the cleaning end 2 into the ear canal. In this case, in order to facilitate the pushing-in of the cleaning end, the end of the cleaning end 2 away from the connecting rod 1 is a round head 6, whose cross section gradually decreases in the direction away from the connecting rod 1 and perpendicular to the length direction of the connecting rod 1, so that the contact area between the section of the cleaning end without the opening of the collecting tank 5 and the earwax in the ear canal can be reduced. With the cooperation of the round head 6 and the collecting tank 5, the possibility that the granular earwax in the ear canal will be pushed towards the outer ear during the ear canal cleaning process can be further reduced.

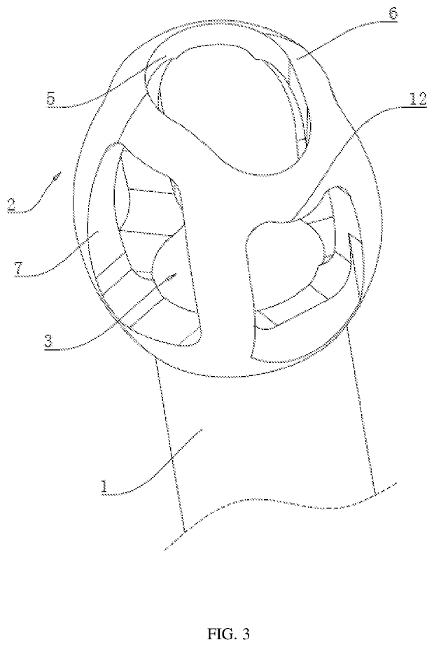

With reference to FIGS. 2 and 3, when removing the earwax in the ear canal, the earpick head must not only remove the fine granular earwax in the ear canal, but also remove the earwax adhering to the inner wall of the ear canal. Therefore, a plurality of lateral grooves 7 in communication with the pre-storage cavity 3 are defined at intervals on the outer wall of the cleaning end 2, and the openings of lateral grooves 7 have different length from each other. In other embodiments, the openings of all the lateral grooves 7 can have the same length. The lateral grooves 7, the collecting tank 5 and the pre-storage cavity 3 are configured, such that the whole cleaning end 2 is hollowed out, and when the earpick head rotates in the ear canal, the inner walls of the lateral groove 7 at two opposite sides in the direction perpendicular to the length direction of the connecting rod 1 can scrape off the earwax on the inner wall of the ear canal, so that the earwax on the inner wall of the ear canal can be removed.

A protrusion 12 is integrally formed on the inner wall of one of the lateral grooves 7 at a side facing the connecting rod 1, and one end of the protrusion 12 facing the connecting rod 1 is an arc surface. The protrusion 12 is provided, such that the function of a normal earpick can be achieved without rotating the connecting rod 1 to drive the cleaning end 2 to rotate during the use of the earpick head.

Referring to FIGS. 2 and 3, when the earwax on the inner wall of the ear canal is scraped off by the inner wall of the lateral groove 7, the scraped earwax enters the pre-storage cavity 3 with the rotation of the cleaning end 2, but a thickness of the inner wall of the lateral groove 7 would hinder the scraped earwax to fall into the pre-storage cavity 3. In order to reduce the probability of the scraped earwax being hindered, a distance between the inner walls on opposite sides of the lateral groove 7 is so determined, that it gradually increases in the direction towards the central axis of the pre-storage cavity 3, so as to reduce the thickness of the inner wall at the opening of the lateral groove 7, thereby reducing the hindering effect on the scraped earwax to enter the pre-storage cavity 3 by the inner wall of the lateral groove 7.

In addition, the maximum cross-sectional diameter of the cleaning end 2 in the direction perpendicular to the connecting rod 1 is set to be greater than the diameter of the connecting rod 1, such that when the cleaning end rotates in the ear canal, the outer wall of the cleaning end 2 contacts the inner wall of the ear canal, which can reduces the probability that the connecting rod 1 contacts the inner wall of the ear canal, such that the inner wall of the ear canal can be protected. At the same time, a connecting bar 41 is formed between the adjacent lateral grooves 7, wherein the maximum distance between the inner walls of the lateral groove 7 in the direction perpendicular to the length direction of the connecting rod 1 is greater than the width of the connecting bar 41. The earwax adhering to the inner wall of the ear canal is removed through the inner wall at the opening of the lateral groove 7, and the width of the lateral groove 7 is designed to be greater than the width of the connecting bar 41, such that the contact area between the outer wall of the cleaning end 2 and the inner wall of the ear canal can be reduced, so that the inner wall of the ear canal can be further protected.

During the use of the earpick head, it is necessary to assemble the earpick head with other fittings on the earpick head. In order to facilitate the assembly of the whole earpick head, a mounting sleeve 8 is connected to one end of the connecting rod 1 away from the cleaning end 2, the mounting sleeve 8 is cylindrical, and the diameter of the mounting sleeve is greater than the diameter of the connecting rod 1. The mounting sleeve 8 is configured with a connecting slot 9 in communication with the transportation cavity 4, the connecting slot extends through the mounting sleeve. At the same time, in order to enhance the security between the mounting sleeve 8 and the connecting rod 1, a trumpet-like transition section 10 is integrally formed between the mounting sleeve 8 and the connecting rod 1, wherein the end of the transition section 10 with a smaller diameter is connected to the connecting rod 1, while the end of the transition section 10 with a larger diameter is connected to the mounting sleeve 8. A transition groove 11 for connecting the connecting slot 9 and the transportation cavity 4 is configured through the transition section 10, and one end of the connecting rod 1 away from the cleaning end 2 can be embedded in the transition groove 11 with interference fit, such that the connecting rod 1 is detachable from the mounting sleeve 8. When a user uses the earpick head as an earpick, he can separate the connecting rod 1 from the mounting sleeve 8, which is convenient to hold. In addition, when assembling the connecting rod 1, in order to make it possible to limit the insertion length of the connecting rod 1 in the transition groove 11 and to position the connecting rod 1 in the transition groove 11, an abutment ring 13 is formed on the inner wall of the transition groove 11 away from the cleaning end 2 in a direction close to the central axis of the transition groove 11, and an end surface of the abutment ring 13 away from the mounting sleeve 8 abuts against an end surface of the connecting rod 1 away from the cleaning end 2.

The implementation principle of the hollowed-out earpick head in the embodiments of the present application is as follows. When the earpick head is used to remove the earwax in the ear canal, the cleaning end 2 is slowly pushed into the ear canal by means of the connecting rod 1, at the same time, the cleaning end 2 is driven to rotate by the connecting rod 1. During the advancement of the cleaning end 2, the granular earwax in the ear canal enters the pre-storage cavity 3 through the opening of the collecting tank 5, and the cleaning end 2 rotates in the ear canal, which leads to the change of the opening direction of the collecting tank 5 in the ear canal. During the rotation of the opening direction of the collecting tank 5, the granular earwax from multiple directions in the ear canal can be sent to the pre-storage cavity 3. At the same time, the inner wall of the lateral groove 7 scrapes off the earwax adhering to the inner wall of the ear canal in the rotation direction of the cleaning end 2 and sends the same into the pre-storage cavity 3. In addition, the transportation cavity 4 is configured, such that the earpick can collect the earwax entering the pre-storage cavity 3 in the earpick through the transportation cavity 4 by means of the negative pressure adsorption, which has the function of sending the earwax out of the pre-storage cavity 3 in time. By the above-mentioned process, when removing the earwax, it is possible to reduce the possibility that the granular earwax in the ear canal is pushed to the outer ear during the cleaning process of the ear canal.

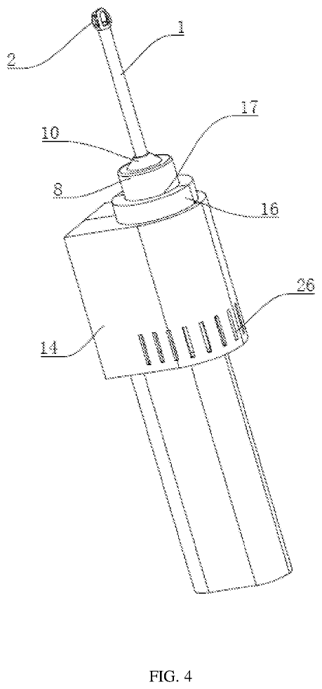

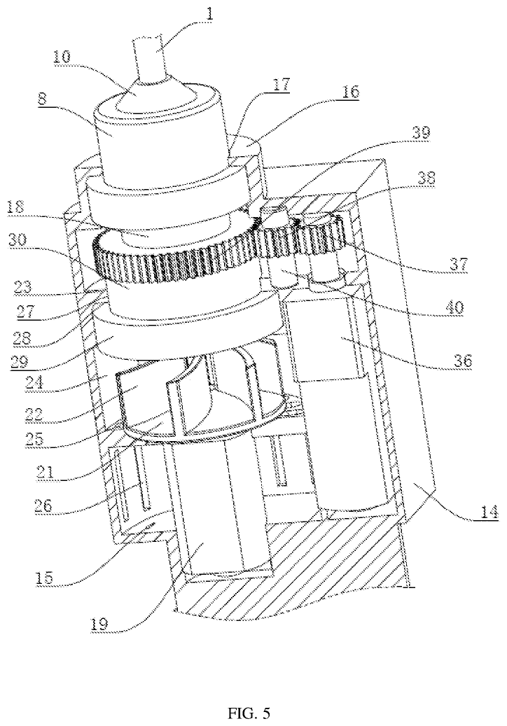

Embodiment I: an earpick using the earpick head is further disclosed. Referring to FIGS. 4 and 5, the earpick includes an earpick head and a housing 14, wherein the earpick head is mounted on the housing 14, the housing 14 is hollowed out with an inner cavity 15, a mounting portion 16 is convexly provided at one end of the housing 14 close to the earpick head and is configured with a mounting port 17 for mounting the earpick head on the housing 14, such that a rotary sleeve 18 capable of being embedded in the connecting slot 9 is arranged in the mounting port 17. After the rotary sleeve 18 is embedded, an outer wall of the rotary sleeve 18 is in an interference fit with an inner wall of the connecting slot 9 to fix the mounting portion 16 and the rotary sleeve 18.

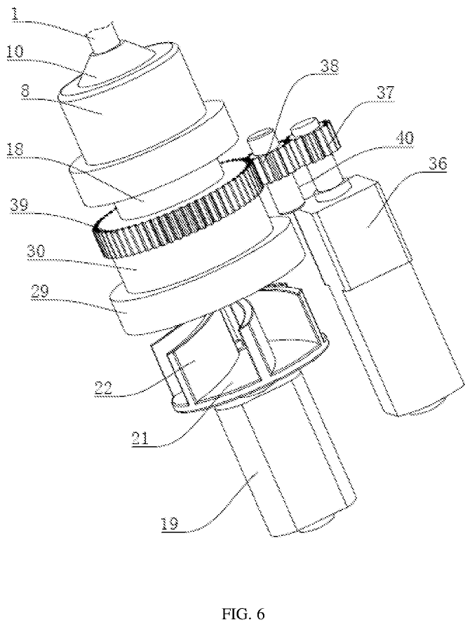

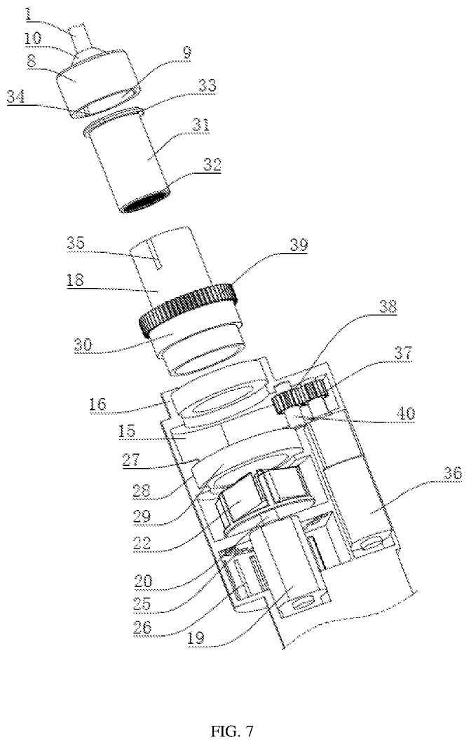

Referring to FIGS. 6 and 7, in order to further ensure that the earpick head can be driven to rotate when the rotary sleeve 18 rotates, the inner wall of the connecting slot 9 is formed with a insertion rib 34, the outer wall of the rotary sleeve 18 is configured with a snapping groove 35 for accommodating the insertion rib 34. During using the earpick, in order to achieve the adsorption function of the earpick and the function of automatically driving the earpick head to rotate, an adsorption mechanism for adsorbing the air of the transportation cavity 4 is arranged inside the inner cavity 15, and a driving mechanism for driving the rotary sleeve 18 to rotate is arranged inside the inner cavity 15, so that the rotary sleeve 18 can be driven to rotate, to drive the earpick head to rotate.

Referring to the embodiment shown in FIGS. 5 and 7, the adsorption mechanism includes an electric motor 19, a rotary shaft 20, a rotary disk 21 and fan blades 22, wherein the electric motor 19 is mounted on the inner wall at the side of the inner cavity 15 away from the earpick head, the rotary shaft 20 is fixedly connected to an output shaft of the electric motor 19, the rotary disk 21 is integrally formed on an end surface of the end of the rotary shaft 20 away from the electric motor 19, the fan blades 22 are arranged on the end of the rotary disk 21 away from the rotary shaft 20. In this embodiment, the number of the fan blades 22 on the rotary disk 21 is six. Besides, a limit portion 23 is formed inside the inner cavity 15, a negative pressure cavity 24 configured for rotation of the rotary disk 21 and the fan blade 22 to form a negative pressure is defined at the limit portion 23, wherein one end of the negative pressure cavity 24 close to the earpick head is aligned with and in communication with the rotary sleeve 18, an inner wall at the side of the negative pressure cavity 24 away from the rotary sleeve 18 is configured with a vent hole 25, an outer wall of the housing 14 is configured with a vent 26 in communication with the inner cavity 15, the air in the housing 14 can be discharged through the vent 26 and the vent hole 25.

With reference to FIGS. 6 and 7, a side of the limit portion 23 close to the earpick head is configured with a mounting groove 27, through which the rotary sleeve 18 can be inserted into the negative pressure cavity 24. A side wall of the mounting groove 27 is configured with a limit groove 28, a first bearing 29 is fixed in the limit groove 28, and the rotary sleeve 18 is extends through the first bearing 29 to facilitate rotation of the rotary sleeve 18. In order to position the rotary sleeve 18, a limit ring 30 is integrally formed on an outer wall of the rotary sleeve 18 at the mounting groove 27, and the limit ring 30 abuts against the side of the first bearing 29 away from the electric motor 19, such that it is possible to limit the insertion length of the rotary sleeve 18 in the negative pressure cavity 24 during assembling the rotary sleeve 18, so as to avoid the direct contact between the rotary sleeve 18 and the fan blades 22, which affects the normal rotation of the fan blades 22.

In addition, when the fan blades 22 rotate, the rotary sleeve 18 transports the earwax removed by the earpick head out of the ear canal. In order to first collect the removed earwax, a collecting sleeve 31 is mounted in one end of the rotary sleeve 18 away from the negative pressure cavity 24, and a grid 32 in shape of a filter mesh is integrally formed at an end opening of the collecting sleeve 31 close to the negative pressure cavity 24. The air circulation between the earpick head and the housing 14 is not affected by the grid 32. The removed earwax can be stored in the collecting sleeve 31, which can be cleaned subsequently. In order to position the collecting sleeve 31 on the rotary sleeve 18, a convex edge 33 is integrally formed on an outer wall at an end of the collecting sleeve 31 away from the grid 32. When the collecting sleeve 31 is embedded in the rotary sleeve 18, the convex edge 33 can abut against an end surface at an end of the rotary sleeve 18 away from the negative pressure cavity 24. The earpick head can be directly removed after once cleaning the ear canal, and then the collecting sleeve 31 can be taken out from the rotary sleeve 18, so that the earwax in the collecting sleeve 31 can be removed.

In this embodiment, the driving mechanism includes a gear motor 36, a first gear 37, a second gear 38 and a third gear 39, wherein the gear motor 36 is mounted in the inner cavity 15, and the gear motor 36 and the limit portion 23 are arranged side by side. The first gear 37 is fixedly sleeved on the output shaft of the gear motor 36, the second gear 38 and the first gear 37 are meshed with each other. A stationary shaft 40 extends through the second gear 38 along its central axis, and two ends of the stationary shaft 40 are respectively fixed on the limit portion 23 and the inner wall of the inner cavity 15. The second gear 38 is rotatable on the stationary shaft 40, the third gear 39 is sleeved on the rotary sleeve 18 and fixed circumferentially, and the third gear 39 and the second gear 38 are meshed with each other.

The implementation principle of the earpick in the embodiment of the present application is as follows. During the use of the earpick, the earpick head is slowly pushed into the ear canal, hereby the electric motor 19 drives the rotary shaft 20 to rotate, and the gear motor 36 drives the first gear 37 to rotate. Since the first gear 37 and the second gear 38 are meshed with each other, and the second gear 38 and the third gear 39 are meshed with each other, the first gear 37 can drive the third gear 39 to rotate, so as to drive the rotary sleeve 18 to rotate, when the first gear 37 rotates. Because of the interference fit between the outer wall of the rotary sleeve 18 with the inner wall of the connecting slot 9 and the engagement between the insertion rib 34 on the inner wall of the connecting slot 9 and the snapping groove 35 at the outer wall of the rotary sleeve 18, the earpick head can rotate automatically when it is slowly pushed into the ear canal, at the same time, the rotary shaft 20 drives the rotary disk 21 and the fan blades 22 to rotate, so that the air in the housing 14 and the negative pressure cavity 24 is discharged through the vent hole 25 and the vent 26, to form a negative pressure in the transportation cavity 4.

In the process that the earpick head is pushed into the ear canal, the rotary sleeve 18 can be driven to rotate in the ear canal through the driving mechanism. During the advancement of the cleaning end 2, the granular earwax in the ear canal enters the pre-storage cavity 3 through the opening of the collecting tank 5, and the cleaning end 2 rotates in the ear canal, which leads to change of the opening direction of the collecting tank 5 in the ear canal. When the opening direction of the collecting tank 5 is rotated, the granular earwax from multiple directions in the ear canal can be sent to the pre-storage cavity 3. At the same time, the inner wall of the lateral groove 7 can scrape off the earwax on the inner wall of the ear canal and send the same to the pre-storage cavity 3, such that the earwax in the ear canal is collected in the pre-storage cavity 3. Because the transportation cavity 4 is in a negative pressure state, the earwax can be transported. Through the above-mentioned process, when the earpick is used to clean off the earwax in the ear canal, the possibility that the granular earwax in the ear canal is pushed to the outer ear during the cleaning process of the ear canal can be reduced, and the removed earwax can be timely discharged from the ear canal.

### Embodiment II

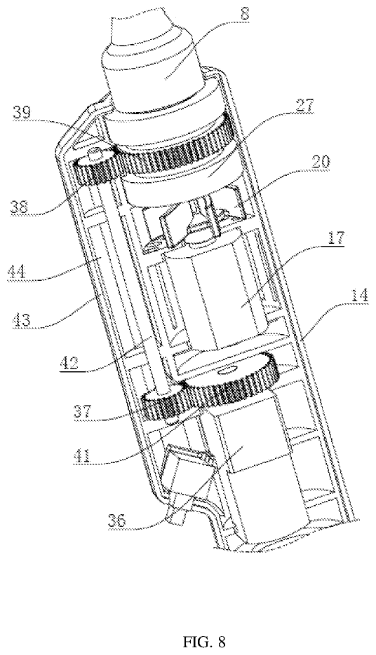

Referring to the embodiment shown in FIG. 8, which differs from the embodiment I in that an additional layer 43 is formed on the outer wall of the housing 14, a different driving mechanism is provided, and a cavity 44 is formed in the additional layer. In this embodiment, the driving mechanism includes a gear motor 36, a driving gear 41, a linkage shaft 42, a second gear 38 and a third gear 39, wherein the gear motor 36 is mounted in the inner cavity 15 and is located on an end of the electric motor 19 away from the earpick head. The driving gear 41 is fixedly sleeved on an output shaft of the gear motor 36, the first gear 37 and the driving gear 41 are meshed with each other. The linkage shaft 42 extends through the first gear 37 at its central axis, one end of the linkage shaft 42 is fixedly connected to the first gear 37, the second gear 38 is fixedly sleeved on an end of the linkage shaft 42 away from the first gear 37, the third gear 39 is sleeved on the rotary sleeve 18 and is fixed circumferentially, and the third gear 39 and the second gear 38 are meshed with each other. After the gear motor 36 is started, the driving gear 41 is driven to rotate, such that the first gear 37 can be driven to rotate because of the engagement of the first gear 37 and the driving gear 41. When the first gear 37 rotates, the linkage shaft 42 can be driven to rotate, thereby driving the second gear 38 to rotate synchronously with the first gear 37, and the second gear 38 and the third gear 39 rotate. When the second gear 38 rotates, the third gear 39 can be driven to rotate, thereby driving the rotary sleeve 18 to rotate.

The above are the preferred embodiments of this application, which are not to limit the protection scope of this application. Therefore, all equivalent changes made according to the structure, shape and principle of this application should be covered by the protection scope of this application.

### LIST OF REFERENCE SIGNS

1 connecting rod
2 cleaning end
3 pre-storage cavity
4 transportation cavity
5 collecting tank
6 round head
7 lateral groove
8 mounting sleeve
9 connecting slot
10 transition section
11 transition groove
12 protrusion
13 abutment ring
14 housing
15 inner cavity
16 mounting portion
17 mounting port
18. rotary sleeve
19 electric motor
20. rotary shaft
21. rotary disk
22 fan blade
23 limit portion
24 negative pressure cavity
25 vent hole
26 vent
27 mounting groove
28 limit groove
29 first bearing
30 limit ring
31 collecting sleeve
32 grid
33. convex edge
34. insertion rib
35 snapping groove
36. gear motor
37 first gear
38 second gear
39 third gear
40 stationary shaft
41 driving gear
42 linkage shaft
43 additional layer
44 cavity

What is claimed is:

1. A hollowed-out earpick head, comprising a connecting rod and a cleaning end on an end surface of one end of the connecting rod, wherein the cleaning end is hollowed out with a pre-storage cavity, the connecting rod is configured with a transportation cavity in communication with the pre-storage cavity, a first end of the cleaning end away from the connecting rod is configured with a collecting tank in communication with the pre-storage cavity, and the cleaning end is hollowed out and in communication with the transportation cavity.

2. The hollowed-out earpick head according to claim 1, wherein the cleaning end is hollowed out with a lateral groove in communication with an outer wall of the pre-storage cavity, and a central axis of the collecting tank is not collinear with a central axis of the connecting rod.

3. The hollowed-out earpick head according to claim 1, wherein the cleaning end is hollowed out with a lateral groove, a protrusion is formed on an inner wall at a side of the lateral groove facing the connecting rod, and an end of the protrusion facing the connecting rod is an arc surface.

4. The hollowed-out earpick head according to claim 1, wherein the first end of the cleaning end away from the connecting rod is a round head, and the round head has a cross section tapering in a direction away from the connecting rod and perpendicular to a length direction of the connecting rod.

5. The hollowed-out earpick head according to claim 1, wherein the cleaning end is hollowed out with a lateral groove, and a distance between an inner wall on opposite sides of the lateral groove gradually increases towards a central axis of the pre-storage cavity.

6. The hollowed-out earpick head according to claim 1, wherein a mounting sleeve is attached to a second end of the connecting rod away from the cleaning end and has a diameter greater than a diameter of the connecting rod, the mounting sleeve is configured with a connecting slot in communication with the transportation cavity, and the connecting slot extends through the transportation cavity.

7. An earpick, comprising a housing and the hollowed-out earpick head according to claim 1, wherein an inner cavity is defined in the housing, an end of the housing close to the hollowed-out earpick head is configured with a mounting port, a rotary sleeve configured for inserting in a connecting slot of a mounting sleeve attached to a second end of the connecting rod away from the cleaning end is provided in the mounting port, an outer wall of the rotary sleeve is circumferentially fixed relative to an inner wall of the connecting slot, an adsorption mechanism configured for adsorbing air in the transportation cavity is provided in the inner cavity, and a driving mechanism configured for driving the rotary sleeve to rotate is provided in the inner cavity.

8. The earpick according to claim 7, wherein the adsorption mechanism comprises an electric motor in the inner cavity, a rotary shaft at an output shaft of the electric motor, a rotary disk at an end of the rotary shaft away from the electric motor, and a fan blade on an end of the rotary disk away from the rotary shaft, a limit portion is provided in the inner cavity and is configured with a negative pressure cavity configured for rotation of the rotary disk and the fan blade to form a negative pressure, air circulates between an end of the negative pressure cavity close to the hollowed-out earpick head and the rotary sleeve, an inner wall at a side of the negative pressure cavity away from the rotary sleeve is configured with a vent hole, and an outer wall of the housing is configured with a vent in communication with the inner cavity.

9. The earpick according to claim 8, wherein a side of the limit portion close to the hollowed-out earpick head is configured with a mounting groove, the rotary sleeve is inserted in the negative pressure cavity through the mounting groove, a limit groove is defined on a side wall of the mounting groove, a first bearing is provided in the limit groove, the rotary sleeve extends through the first bearing, a limit ring is provided on the outer wall of the rotary sleeve at the mounting groove, and the limit ring abuts against a side of the first bearing away from the electric motor.

10. The earpick according to claim 9, wherein a collecting sleeve is provided in an end of the rotary sleeve away from the negative pressure cavity, a grid in a form of a filter mesh is provided at an end port of a first end of the collecting sleeve close to the negative pressure cavity, an outer wall at a second end of the collecting sleeve away from the grid is provided with a convex edge, and the convex edge is configured to abut against an end surface at the end of the rotary sleeve away from the negative pressure cavity.
